(19) United States
(12) Patent Application Publication
(10) Pub. No.: US 2003/0133951 A1
(43) Pub. Date: Jul. 17, 2003

(54) Pharmaceutical composition for treatment of acute, chronic pain and/or neuropathic pain and migraines

(75) Inventors: Jotham W. Coe, Niantic, CT (US); Steven B. Sands, Stonington, CT (US); Edmund P. Harrigan, Old Lyme, CT (US); Brian T. O'Neill, Old Saybrook, CT (US); Eric J. Watsky, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/348,381

(22) Filed: Jan. 21, 2003

Related U.S. Application Data
(63) Continuation of application No. 09/740,307, filed on Dec. 18, 2000, now abandoned.
(60) Provisional application No. 60/195,738, filed on Apr. 7, 2000.

(51) Int. Cl. A61K 35/78; A61K 31/57; A61K 31/5513; A61K 31/4709; A61K 31/485; A61K 31/454; A61K 39/08

(52) U.S. Cl. 424/239.1; 514/312; 514/326; 514/317; 514/282; 514/169; 514/221; 514/263.34; 424/760

(57) ABSTRACT

Pharmaceutical compositions are disclosed for the treatment of acute, chronic and/or neuropathic pain. The pharmaceutical compositions are comprised of a therapeutically effective combination of a nicotine receptor partial agonist and an analgesic agent and a pharmaceutically acceptable carrier. The analgesic agent is selected from opioid analgesics, NMDA antagonists, substance P antagonists, COX 1 and COX 2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, and botulinum toxin. The method of using these compounds and a method of treating acute, chronic and/or neuropathic pain and migraine in a mammal including a human is also disclosed.

### BACKGROUND OF THE INVENTION

The present invention relates to pharmaceutical compositions for the treatment of acute, chronic and/or neuropathic pain and migraine in a mammal (e.g. human) comprising a nicotine receptor partial agonist (NRPA) and analgesic agents, including opioid analgesics, NMDA antagonists, substance P antagonists, COX 1 and COX 2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, N-type calcium channel antagonists and botulinum toxin. The term NRPA refers to all chemical compounds which bind at neuronal nicotinic acetylcholine specific receptor sites in mammalian tissue and elicit a partial agonist response. A partial agonist response is defined here to mean a partial, or incomplete functional effect in a given functional assay. Additionally, a partial agonist will also exhibit some degree of antagonist activity by its ability to block the action of a full agonist (Feldman, R. S., Meyer, J. S. & Quenzer, L. F. Principles of Neuropsychopharmacology, 1997; Sinauer Assoc. Inc.). The present invention may be used to treat mammals (e.g. humans) for acute, chronic and/or neuropathic pain with a decrease in the severity of unwanted side effects such as causing nausea and/or stomach upset.

The invention also relates to aryl fused azapolycylic compounds that bind to neuronal nicotinic acetylcholine specific receptor sites and are useful in modulating cholinergic function and are referred to in WO 9818798-A1, WO 9935131-A1 and WO 9955680-A1. The foregoing applications are owned in common with the present application and are incorporated herein by reference in their entireties.

Analgesic agents decrease pain perception. In animal models of pain states, the above compounds inhibit acute pain perception. These compounds also inhibit pain sensitization processes in which the perception of the painfulness of a given stimulus is increased without any change in stimulus intensity. In humans, analgesic agents have also been found to decrease both acute pain perception and sensitization. Opioid analgesic agents, in particular, remain the most effective means of alleviating severe pain across a broad spectrum, including inflammatory as well as neuropathic pain states. However, even though analgesic agents have therapeutic utility in the treatment of pain, there are significant liabilities to the use of analgesic compounds. Specifically, many of these compounds that have been tested in humans can cause potentially serious side effects such as gastrointestinal complications including nausea, emesis, ulcers, and constipation, respiratory depression, and psychological and physical dependence.

### SUMMARY OF INVENTION

The present invention relates to a pharmaceutical composition for the treatment of acute, chronic and/or neuropathic pain and migraine comprising (a) a nicotine receptor partial agonist or a pharmaceutically acceptable salt thereof; (b) an analgesic agent or pharmaceutically acceptable salt thereof and (c) a pharmaceutically acceptable carrier; wherein the active agents “a” and “b” above are present in amounts that render the composition effective in treating acute, chronic and/or neuropathic pain, and migraine

A nicotinic partial agonist combined with an analgesic agent may inhibit pain sensitization and pain perception while reducing the incidence of undesirable side effects. A nicotinic partial agonist combined with an analgesic agent may inhibit pain sensitization and pain perception while reducing the incidence of undesirable side effects. Nicotine has long been appreciated to have antinociceptive properties, but its use has been limited by a poor spectrum of activity, side effects, and less efficacy than opioids. This may be due to a lack of specificity of nicotine for neuromuscular, ganglionic, and central nervous system receptors. The development of nicotine partial agonists with specific receptor subtype affinities is an approach to potentially reduce side effects and enhance efficacy.

In a more specific embodiment of the invention the analgesic agent is selected from opioid analgesics, NMDA antagonists, substance P antagonists, COX 1 and COX 2 inhibitors, tricyclic antidepressants (TCA), selective serotonin reuptake inhibitors, (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, N-type calcium channel antagonists and botulinum toxin.

In another more specific embodiment of this invention, the nicotine receptor partial agonist is selected from:

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-ethyl-1,2,3,4,5,6-hexahydro-1,5-methano-pyrido[1,2-a][1,5]diazocin-8-one;

9-methyl-1,2,3,4,5,6-hexahydro-1,5-methano-pyrido[1,2-a][1,5]diazocin-8-one;

9-phenyl-1,2,3,4,5,6-hexahydro-1,5-methano-pyrido[1,2-a][1,5]diazocin-8-one;

9-vinyl-1,2,3,4,5,6-hexahydro-1,5-methano-pyrido[1,2-a][1,5]diazocin-8-one;

9-bromo-3-methyl-1,2,3,4,5,6-hexahydro-1,5-methano-pyrido[1,2-a][1,5]diazocin-8-one;

3-benzyl-9-bromo-1,2,3,4,5,6-hexahydro-1,5-methano-pyrido[1,2-a][1,5]diazocin-8-one;

3-benzyl-9-chloro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-ethynyl-1,2,3,4,5,6-hexahydro-1,5-methano-pyrido[1,2a][1,5]diazocin-8-one;

9-(2-propenyl)-1,2,3,4,5,6-hexahydro-1,5-methano-pyrido[1,2a][1,5]diazocin-8-one;

9-(2-propyl)-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-⁸-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

9-(4-fluorophenyl)-1,2,3,4,5,6-hexahydro-1,5-methano-pyrido[1,2a][1,5]diazocin-8-one;

9-(3-fluorophenyl)-1,2,3,4,5,6-hexahydro-1,5-methano-pyrido[1,2a][1,5]diazocin-8-one;

9-(3,5-difluorophenyl)-1,2,3,4,5,6-hexahydro-1,5-methano-pyrido[1,2a][1,5]diazocin-8-one;

9-(2,4-difluorophenyl)-1,2,3,4,5,6-hexahydro-1,5-methano-pyrido[1,2a][1,5]diazocin-8-one;

9-(2,5-difluorophenyl)-1,2,3,4,5,6-hexahydro-1,5-methano-pyrido[1,2a][1,5]diazocin-8-one;

6-methyl-5-oxo-6,13-diazatetracyclo[9.3.1.0^{2,10}.0^{4,8}]pentadeca-2(10),3,8-triene;

5-oxo-6,13-diazatetracyclo[9.3.1.0^{2,10}.0^{4,8}]pentadeca-2(10),3,8-triene;

6-oxo-5,7,13-triazatetracyclo[9.3. 1.0^{2,10}.0^{4,8}]pentadeca-2(10),3,8triene;

4,5-difluoro-10-aza-tricyclo[6.3.1.0^{2,7}]dodeca-2(7),3,5-triene;

5-fluoro-10-aza-tricyclo[6.3.1.0^{2,7}]dodeca-2(7),3,5-triene-4-carbonitrile;

4-ethynyl-5-fluoro-10-aza-tricyclo[6.3.1.0^{2,7}]dodeca-2(7),3,5-triene;

5-ethynyl-10-aza-tricyclo[6.3.1.0^{2,7}]dodeca-2(7),3,5-triene-4-carbonitrile;

6-methyl-5-thia-5-dioxa-6,13-diazatetracyclo[9.3.1.0^{2,10}.0^{4,8}]pentadeca-2(10),3,8-triene;

10-aza-tricyclo[6.3.1.0^{2,7}]dodeca-2(7),3,5-triene;

4-fluoro-10-aza-tricyclo[6.3.1.0^{2,7}]dodeca-2(7),3,5-triene;

4-methyl-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

7-methyl-5,7,13-triazatetracyclo[9.3.1.0^{2,10}.0^{4,8}]pentadeca-2(10),3,5,8-tetraene;

6-methyl-5,7,13-triazatetracyclo[9.3.1.0^{2,10}.0^{4,8}]pentadeca-2(10),3,5,8-tetraene;

6,7-dimethyl-5,7,13-triazatetracyclo[9.3.1.0^{2,10}.0^{4,8}]pentadeca-2(10),3,5,8-tetraene;

6-methyl-7-phenyl-5,7,13-triazatetracyclo[9.3.1.0^{2,10}.0^{4,8}]pentadeca-2(10),3,5,8tetraene;

6,7-dimethyl-5,8,14-triazatetracyclo[10.3.1.0^{2,11}.0^{4,9}]hexadeca-2(11),3,5,7,9-pentaene;

5,8,14-triazatetracyclo[10.3.1.0^{2,11}.0^{4,9}]hexadeca-2(11),3,5,7,9-pentaene;

14-methyl-5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

4-chloro-10-azatricyclo[6.3.1.0^{2,7}]dodeca-2(7),3,5-triene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

10-azatricyclo[6.3.1.0^{2,7}]dodeca-2(7),3,5-trien-4-ol;

7-methyl-5-oxa-6,13-diazatetracyclo[9.3.1.0^{2,10}.0^{4,8}]pentadeca-2,4(8),6,9-tetraene;

4,5-dichloro-10-azatricyclo[6.3.1.0^{2,7}]dodeca-2(7),3,5-triene;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

5,7,14-triazatetracyclo[10.3.1.0^{2,10}.0^{4,8}]hexadeca-2(10),3,5,8-tetraene;

5,6-dimethyl-5,7,14-triazatetracyclo[10.3.1.0^{2,10}.0^{4,8}]hexadeca-2(10),3,6,8-tetraene;

5-methyl-5,7,14-triazatetracyclo[10.3.1.0^{2,10}.0^{4,8}]hexadeca-2(10),3,6,8-tetraene;

6-(trifluoromethyl)-7-thia-5,14-diazatetracyclo[10.3.1.0^{2,10}.0^{4,8}]hexadeca-2(10),3,5,8-tetraene;

5,8,15-triazatetracyclo[11.3.1.0^{2,11}.0^{4,9}]heptadeca-2(11),3,5,7,9-pentaene;

7-methyl-5,8,15-triazatetracyclo[11.3.10^{2,11}.0^{4,9}]heptadeca-2(11),3,5,7,9-pentaene;

6-methyl-5,8,15-triazatetracyclo[11.3.1.0^{2,11}.0^{4,9}]heptadeca-2(11),3,5,7,9-pentaene;

6,7-dimethyl-5,8,15-triazatetracyclo[11.3.1.0^{2,11}.0^{4,9}]heptadeca-2(11),3,5,7,9-pentaene;

7-oxa-5,14-diazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

5-methyl-7-oxa-6,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

7-methyl-5-oxa-6,14-diazatetracyclo[10.3.1.0^{2,10}.0^{4,8}]hexadeca-2(10),3,6,8-tetraene;

4,5-difluoro-11-azatricyclo[7.3.1.0^{2,7}]trideca-2(7),3,5-triene;

4-chloro-5-fluoro-11-azatricyclo[7.3.1.0^{2,7}]trideca-2(7),3,5-triene;

5-chloro-4-fluoro-11-azatricyclo[7.3.1.0^{2,7}]trideca-2(7),3,5-triene;

4-(1-ethynyl)-5-fluoro-11-azatricyclo[7.3.1.0^{2,7}]trideca-2(7),3,5-triene;

5-(1-ethynyl)-4-fluoro-11-azatricyclo[7.3.1.0^{2,7}]trideca-2(7),3,5-triene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

11-aza-tricyclo[7.3.1.0^{2,7}]trideca-2(7),3,5-trien-6-ol;

6-fluoro-11-aza-tricyclo[7.3.1.0^{2,7}]trideca-2(7),3,5-triene;

11-aza-tricyclo[7.3.1.0^{2,7}]trideca-2(7),3,5-trien-5-ol;

4-nitro-11-aza-tricyclo[7.3.1.0^{2,7}]trideca-2(7),3,5-triene;

5-nitro-11-aza-tricyclo[7.3.1.0^{2,7}]trideca-2(7),3,5-triene;

5-fluoro-11-aza-tricyclo[7.3.1.0^{2,7}]trideca-2(7),3,5-triene; and

6-hydroxy-5-methoxy-11-aza-tricyclo[7.3.1.0^{2,7}]trideca-2(7),3,5-triene and their pharmaceutically acceptable salts and their optical isomers.

Preferably, the nicotine receptor partial agonist is selected from

b 9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

6-methyl-5-thia-5-dioxa-6,13-diazatetracyclo[9.3.1.0^{2,10}.0^{4,8}]pentadeca-2(10),3,8-triene;

4-fluoro-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

6-methyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,9}]hexadeca-2(11),3,5,7,9-pentaene;

5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10} .0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6 -methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-l 1-aza-tricyclo[7.3.1.0^{2,7}]trideca-2(7),3,5-triene;

6-fluoro-11-aza-tricyclo[7.3.1.0^{2,7}]trideca-2(7),3,5-triene; and

11-aza-tricyclo[7.3.1.0^{2,7}]trideca-2(7),3,5-trien-5-ol; and

their pharmaceutically acceptable salts and their optical isomers.

In a more specific embodiment of the invention, the analgesic is selected from an opioid analgesic, such as propoxyphene (Darvon), meperidine (Demerol), hydromorphone (Dilaudid), hydrocodone (Lortab), morphine, codeine and tramodol; an NMDA antagonist such as dextromethorphan, 2-piperidinol-1-alkanol derivatives as described in the U.S. Pat. No. 5,272,160 and incorporated herein by reference, eliprodil, and ifenprodil; a COX 2 inhibitor such as rofecoxib or celecoxib; a COX 1 inhibitor such as salycylic acid (aspirin), diclofenac, oxicams, indomethacin, ibuprofen, and naproxen; an anticonvulsant, such as gabapentin (Neurontin), carbamazepine, pregabalin, topiramate and valproic acid; a migraine agent such as elitriptan, sumatriptan, rizatriptan, zolmitriptan, and naratriptan; a skeletal muscle relaxant, such as flexeril, carisoprodol (Soma), robaxisal, norgesic and dantrium; benzodiazepines such as diazepam (Valium), chlordiazepoxide (Librium), alprazolam (Xanax) and lorazepam (Ativan); acetominophen; anesthetic agents such as nitrous oxide, halothane, lidocaine, etidocaine, ropivacaine, chloroprocaine, sarapin and bupivacaine; capsaicin receptor agonists such as Arithricare®; and TCAs (tricyclic antidepressants) such as, desipramine, amitriptyline, doxepin, perphenazine, protriptyline and tranylcypromine. In another specific embodiment of this invention the analgesic agent is selected from anti-hypertensives such as clonidine; anti-arrythmics such as mexilitene; antihistamines such as diphenhydraimine and hydroxyzine, caffeine; and steroids such as prednisone, methyl-prednisone and decadron; serotonin uptake blockers such as paroxitine, sertraline and fluoxetine; and levodopa. In another specific embodiment of the invention the analgesic agents is selected from substance P antagonists and N-type calcium channel antagonists such as Ziconotide®.

The invention also relates to a method of treating acute, chronic and/or neuropathic pain and migraine in a mammal comprising administering to said mammal, respectively a pain attenuating effective amount of a pharmaceutical composition comprising: (a) a nicotine receptor partial agonist or a pharmaceutically acceptable salt thereof; (b) an analgesic agent or pharmaceutically acceptable salt thereof and (c) a pharmaceutically acceptable carries, wherein the active agents “a” and “b” above are present in amounts that render the composition effective in treating acute, chronic and/or neuropathic pain and migraine.

In another more specific embodiment of this invention the nicotine receptor partial agonist is selected from

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-ethyl-1,2,3,4,5,6-hexahydro-1,5-methano-pyrido[1,2-a][1,5]diazocin-8-one;

9-methyl-1,2,3,4,5,6-hexahydro-1,5-methano-pyrido[1,2-a][1,5]diazocin-8-one;

9-phenyl-1,2,3,4,5,6-hexahydro-1,5-methano-pyrido[1,2-a][1,5]diazocin-8-one;

9-vinyl-1,2,3,4,5,6-hexahydro-1,5-methano-pyrido[1,2-a][1,5]diazocin-8-one;

9-bromo-3-methyl-1,2,3,4,5,6-hexahydro-1,5-methano-pyrido[1,2-a][1,5]diazocin-8-one;

3-benzyl-9-bromo-1,2,3,4,5,6-hexahydro-1,5-methano-pyrido[1,2-a][1,5]diazocin-8-one;

3-benzyl-9-chloro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-ethynyl-1,2,3,4,5,6-hexahydro-1,5-methano-pyrido[1,2a][1,5]diazocin-8-one;

9-(2-propenyl)-1,2,3,4,5,6-hexahydro-1,5-methano-pyrido[1,2a][1,5]diazocin-8-one;

9-(2-propyl)-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

9-(4-fluorophenyl)-1,2,3,4,5,6-hexahydro-1,5-methano-pyrido[1,2a][1,5]diazocin-8-one;

9-(3-fluorophenyl)-1,2,3,4,5,6-hexahydro-1,5-methano-pyrido[1,2a][1,5]diazocin-8-one;

9-(3,5-difluorophenyl)-1,2,3,4,5,6-hexahydro-1,5-methano-pyrido[1,2a][1,5]diazocin-8-one;

9-(2,4-difluorophenyl)-1,2,3,4,5,6-hexahydro-1,5-methano-pyrido[1,2a][1,5]diazocin-8-one;

9-(2,5-difluorophenyl)-1,2,3,4,5,6-hexahydro-1,5-methano-pyrido[1,2a][1,5]diazocin-8-one;

6-methyl-5-oxo-6,13-diazatetracyclo[9.3.1.0^{2,10}.0^{4,8}]pentadeca-2(10),3,8-triene;

5-oxo-6,13-diazatetracyclo[9.3.1.0^{2,10}.0^{4,8}]pentadeca-2(10),3,8-triene;

6-oxo-5,7,13-triazatetracyclo[9.3.1.0^{2,10}.0^{4,8}]pentadeca-2(10),3,8-triene;

4,5-difluoro-10-aza-tricyclo[6.3.1.0^{2,7}]dodeca-2(7),3,5-triene;

5-fluoro-10-aza-tricyclo[6.3.1.0^{2,7}]dodeca-2(7),3,5-triene-4-carbonitrile;

4-ethynyl-5-fluoro-10-aza-tricyclo[6.3.1.0^{2,7}]dodeca-2(7),3,5-triene;

5-ethynyl-10-aza-tricyclo[6.3.1.0^{2,7}]dodeca-2(7),3,5-triene-4-carbonitrile;

6-methyl-5-thia-5-dioxa-6,13-diazatetracyclo[9.3.1.0^{2,10}.0^{4,8}]pentadeca-2(10),3,8-triene;

10-aza-tricyclo[6.3.1.0^{2,7}]dodeca-2(7),3,5-triene;

4-fluoro-10-aza-tricyclo[6.3.1.0^{2,7}]dodeca-2(7),3,5-triene;

4-methyl-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

7-methyl-5,7,13-triazatetracyclo[9.3.1.0^{2,10}.0^{4,8}]pentadeca-2(10),3,5,8-tetraene;

6-methyl-5,7,13-triazatetracyclo[9.3.1.0^{2,10}.0^{4,8}]pentadeca-2(10),3,5,8-tetraene;

6,7-dimethyl-5,7,13-triazatetracyclo[9.3.1.0^{2.10}.0^{4,8}]pentadeca-2(10),3,5,8-tetraene;

6-methyl-7-phenyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,9}]hexadeca-2(11),3,5,7,9-pentaene;

5,8,1 4-triazatetracyclo[10.3.1.0^{2,11}.0^{4,9}]hexadeca-2(11),3,5,7,9-pentaene;

14-methyl-5,8,1 4-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

4-chloro-10-azatricyclo[6.3.1.0^{2,7}]dodeca-2(7),3,5-triene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

10-azatricyclo[6.3.1.0^{2,7}]dodeca-2(7),3,5-trien-4-ol;

7-methyl-5-oxa-6,13-diazatetracyclo[9.3.1.0^{2,10}.0^{4,8}]pentadeca-2,4(8),6,9-tetraene;

4,5-dichloro-10-azatricyclo[6.3.1.0^{2,7}]dodeca-2(7),3,5-triene;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

5,7,14-triazatetracyclo[10.3.1.0^{2,10}.0^{4,8}]hexadeca-2(10),3,5,8-tetraene;

5,6-dimethyl-5,7,14-triazatetracyclo[10.3.1.0^{2,10}.0^{4,8}]hexadeca-2(10),3,6,8-tetraene;

5-methyl-5,7,14-triazatetracyclo[10.3.1.0^{2,10}.0^{4,8}]hexadeca-2(10),3,6,8-tetraene;

6-(trifluoromethyl)-7-thia-5,14-diazatetracyclo[10.3.1.0^{2,10}.0^{4,8}]hexadeca-2(10),3,5,8-tetraene;

5,8,15-triazatetracyclo[11.3.1.0^{2,11}.0^{4,9}]heptadeca-2(11),3,5,7,9-pentaene;

7-methyl-5,8,15-triazatetracyclo[11.3.1.0^{2,11}.0^{4,9}]heptadeca-2(11),3,5,7,9-pentaene;

6-methyl-5,8,15-triazatetracyclo[11.3.1.0^{2,11}.0^{4,9}]heptadeca-2(11),3,5,7,9-pentaene;

6,7-dimethyl-5,8,15-triazatetracyclo[11.3.1.0^{2,11}.0^{4,9}]heptadeca-2(11),3,5,7,9-pentaene;

7-oxa-5,14-diazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

5-methyl-7-oxa-6,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

7-methyl-5-oxa-6,14-diazatetracyclo[10.3.1.0^{2,10}.0^{4,8}]hexadeca-2(10),3,6,8-tetraene;

4,5-difluoro-11-azatricyclo[7.3.1.0^{2,7}]trideca-2(7),3,5-triene;

4-chloro-5-fluoro-11-azatricyclo[7.3.1.0^{2,7}]trideca-2(7),3,5-triene;

5-chloro-4-fluoro-11-azatricyclo[7.3.1.0^{2,7}]trideca-2(7),3,5-triene;

4-(1-ethynyl)-5-fluoro-11-azatricyclo[7.3.1.0^{2,7}]trideca-2(7),3,5-triene;

5-(1-ethynyl)-4-fluoro-11-azatricyclo[7.3.1.0^{2,7}]trideca-2(7),3,5-triene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

11-aza-tricyclo[7.3.1.0^{2,7}]trideca-2(7),3,5-trien-6-ol;

6-fluoro-11-aza-tricyclo[7.3.1.0^{2,7}]trideca-2(7),3,5-triene;

11-aza-tricyclo[7.3.1.0^{2,7}]trideca-2(7),3,5-trien-5-ol;

4-nitro-11-aza-tricyclo[7.3.1.0^{2,7}]trideca-2(7),3,5-triene;

5-nitro-11-aza-tricyclo[7.3.1.0^{2,7}]trideca-2(7),3,5-triene;

5-fluoro-11-aza-tricyclo[7.3.1.0^{2,7}]trideca-2(7),3,5-triene; and

6-hydroxy-5-methoxy-11-aza-tricyclo[7.3.1.0^{2,7}]trideca-2(7),3,5-triene and their pharmaceutically acceptable salts and their optical isomers.

Preferably, the nicotine receptor partial agonist is selected from

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

6-methyl-5-thia-5-dioxa-6,13-diazatetracyclo[9.3.1.0^{2,10}.0^{4,8}]pentadeca-2(10),3,8-triene;

4-fluoro-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

6-methyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,9}]hexadeca-2(11),3,5,7,9-pentaene;

5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

6-fluoro-11-aza-tricyclo[7.3.1.0^{2,7}]trideca-2(7),3,5-triene; and

11-aza-tricyclo[7.3.1.0^{2,7}]trideca-2(7),3,5-trien-5-ol; and the pharmaceutically acceptable salts and optical isomers of the foregoing compounds.

In a more specific embodiment the TCA analgesic agents are selected from doxepin, desipramine, trimipramine, perphenazine, protriptyline and tranylcypromine. In another more specific embodiment the anesthetic agents are selected from nitrous oxide, halothane, lidocaine, etidocaine, ropivacaine, chloro-procaine, sarapin and bupivacaine. In another more specific embodiment the benzodiazepine analgesic agents are selected from diazepam, chlordiazepoxide, alprazolam and lorazepam. In another more specific embodiment the skeletal muscle relaxant analgesic agents are selected from flexeril, carisoprodol, robaxisal, norgesic and dantrium. In yet another more specific embodiment the migraine therapeutic agents are selected from elitriptan, sumatriptan, rizatriptan, zolmitriptan and naratriptan. In yet another more specific embodiment the anticonvulsant analgesic agents are selected from gabapentin, carbamazepine, topiramate, valproic acid and pregabalin. In yet another more specific embodiment the opioid analgesic agent is selected from propoxyphene, meperidine, hydro-morphone, hydrocodone, morphine, codeine and tramadol. In yet another more specific embodiment the NMDA antagonists are selected from dextromethorphan, 2-piperidinol-1-alkanol derivatives as described in the U.S. Pat. No. 5,272,160, eliprodil ifenprodil. In yet another more specific embodiment the COX 2 inhibitor analgesic agents are selected from rofecoxib and celecoxib. In yet another more specific embodiment the COX 1 inhibitor analgesic agents are selected from salycylic acid, acetominophen, diclofenac, baclofen, piroxicam, indomethacin, ibuprofen, and naproxen. In yet another specific embodiment the analgesic agents are selected from clonidine, mexilitene, diphenhydramine, hydroxyzine, caffeine, prednisone, methylprednisolone and decadron. In yet another specific embodiment the analgesic agents are selected from fluoxetine, sertraline and paroxetine. In yet another specific embodiment the analgesic agent is levodopa, Ziconotide® and substance P antagonists.

This invention also relates to a pharmaceutical composition for treating a disorder or condition selected from the group consisting of diseases and conditions in which pain predominates, including acute pain, chronic pain, neuropathic pain and migraine, and including soft tissue and peripheral damage, such as acute trauma, osteoarthritis, rheumatoid arthritis, musculo-skeletal pain, particularly after trauma, spinal pain, dental pain, myofascial pain syndromes, headache, episiotomy pain, and burns; deep and visceral pain, such as heart pain, muscle pain, eye pain, orofacial pain, for example, odontaigia, abdominal pain, gynaecological pain, for example, dysmenorrhea, and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; headache, including migraine, acute or chronic tension headache, cluster headache, temporomandibular pain and maxillary sinus pain; ankylosing spondylitis, gout; post operative pain; and scar pain, in a mammal, including a human, the method comprising administering to said mammal respectively a pain attenuating effective amount of a pharmaceutical composition comprising: (a) a nicotine receptor partial agonist or a pharmaceutically acceptable salt thereof; (b) an analgesic agent or a pharmaceutically acceptable salt thereof and (c) a pharmaceutically acceptable carrier, wherein the active agents “a” and “b” above are present in amounts that render the composition effective in treating acute, chronic and/or neuropathic pain and migraine.

This invention also relates to a method of treating a disorder or condition selected from the group consisting of diseases and conditions in which pain predominates, including acute pain, chronic pain, neuropathic pain and migraine, and including soft tissue and peripheral damage, such as acute trauma, osteoarthritis, rheumatoid arthritis, musculo-skeletal pain, particularly after trauma, spinal pain, dental pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhea, and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; headache, including migraine, acute or chronic tension headache, cluster headache, temporomandibular pain and maxillary sinus pain; ankylosing spondylitis, gout; post operative pain; and scar pain, in a mammal, including a human, the method comprising administering to said mammal respectively a pain attenuating effective amount of a pharmaceutical composition comprising: (a) a nicotine receptor partial agonist or a pharmaceutically acceptable salt thereof; (b) an analgesic agent or a pharmaceutically acceptable salt thereof and (c) a pharmaceutically acceptable carrier, wherein the active agents “a” and “b” above are present in amounts that render the composition effective in treating acute, chronic and/or neuropathic pain and migraine.

The term “treating” as used herein, refers to reversing, alleviating, inhibiting or slowing the progress of, or preventing the disorder or condition to which such term applies, or one or more symptoms of such disorder or condition. The term “treatment”, as used herein, refers to the act of treating, as “treating” is defined immediately above.

The chemist of ordinary skill will recognize that certain compounds of this invention will contain one or more atoms which may be in a particular stereochemical or geometric configuration, giving rise to stereoisomers and configurational isomers. All such isomers and mixture thereof are included in this invention. Hydrates of the compounds of this invention are also included.

The chemist of ordinary skill will recognize that certain combinations of heteroatom-containing substituents listed in this invention define compounds which will be less stable under physiological conditions (e.g., those containing acetal or animal linkages). According, such compounds are less preferred.

### DETAILED DESCRIPTION OF THE INVENTION

In combination with the NRPA, the invention includes an analgesic agent or a pharmaceutically acceptable salt of compounds such as opioid analgesics, NMDA antagonists, substance P antagonists, COX 1 and COX 2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, N-type calcium channel antagonists and botulinum toxin. The herein below references refer, collectively, to quinuclidine, piperidine, ethylene diamine, pyrrolidine and azanorbornane derivatives and related compounds that exhibit activity as substance P receptor antagonists and that can be used, in the pharmaceutical compositions and methods of this invention, and to methods of preparing the same: U.S. Pat. No. 5,162,339, which issued on Nov. 11, 1992; U.S. Pat. No. 5,232,929, which issued on Aug. 3, 1993; World Patent Application WO 92/20676, published Nov. 26, 1992; World Patent Application WO 93/00331, published Jan. 7, 1993; World Patent Application WO 92/21677, published Dec. 10, 1992; World Patent Application WO 93/00330, published Jan. 7, 1993; World Patent Application WO 93/06099, published Apr. 1, 1993; World Patent Application WO 93/10073, published May 27, 1993; World Patent Application WO 92/06079, published Apr. 16,1992; World Patent Application WO 92/12151, published Jul. 23,1992; World Patent Application WO 92/15585, published Sep. 17, 1992; World Patent Application WO 93/10073, published May 27, 1993; World Patent Application WO 93/19064, published Sep. 30,1993; World Patent Application WO 94/08997, published Apr. 28, 1994; World Patent Application WO 94/04496, published Mar. 3, 1994; World Patent Application WO 95/07908, published Mar. 3, 1995; World Patent Application WO 94/20500, published Sep. 15, 1994; World Patent Application WO 94/13663, published Jun. 23, 1994; World Patent Application WO 95/16679, published Jun. 22, 1995; World Patent Application WO 97/08144, published Mar. 6, 1997; World Patent Application WO 97/03066, published Jan. 30, 1997; World Patent Application WO 99/25714, published May 27, 1999; U.S. patent application Ser. No. 988,653, filed Dec. 10, 1992; U.S. patent application Ser. No. 026,382, filed Mar. 4, 1993; U.S. patent application Ser. No. 123,306, filed Sep. 17, 1993, and U.S. patent application Ser. No. 072,629, filed Jun. 4, 1993. All of the foregoing World Patent Applications designate the United States. The foregoing patents and patent applications are incorporated herein by reference in their entirety.

Other substance P receptor antagonists that can be used, in the pharmaceutical compositions and methods of this invention are those compounds and pharmaceutically acceptable salts described in the following references: European Patent Application EP 499,313, published Aug. 19, 1992; European Patent Application EP 520,555, published Dec. 30, 1992; European Patent Application EP 522,808, published Jan. 13, 1993, European Patent Application EP 528,495, published Feb. 24, 1993, PCT Patent Application WO 93/14084, published Jul. 22, 1993, PCT Patent Application WO 93/01169, published Jan. 21, 1993, PCT Patent Application WO 93/01165, published Jan. 21, 1993, PCT Patent Application WO 93/01159, published Jan. 21, 1993, PCT Patent Application WO 92/20661, published Nov. 26, 1992, European Patent Application EP 517,589, published Dec. 12, 1992, European Patent Application EP 428,434, published May 22, 1991, and European Patent Application EP 360,390, published Mar. 28, 1990, WO 94/13663 published Jun. 23, 1994; WO 97/08144 published Mar. 6, 1997; WO 97/03066 published Jan. 30, 1997; WO 99/125714 published May 27, 1999; WO 94/20500 published Sep. 15, 1994; WO 93/003300 published Jan. 7, 1993; and U.S. Provisional Patent No. 60/164,692 application filed Nov. 10, 1999. All of the foregoing World Patent Applications designate the United States. The foregoing patents and patent applications are incorporated herein by reference in their entirety. The particular NRPA compounds listed above, which can be employed in the method and pharmaceutical, compositions of this invention, can be made by processes known in the chemical arts, for example by the methods described in WO 9818798 A1, WO 9935131-A1 and U.S. Provisional Patent Application No. 60/083,556 filed Apr. 29, 1998. Some of the preparation methods useful for making the compounds of this invention may require protection of remote functionality (i.e., primary amine, secondary amine, carboxyl). The need for such protection will vary depending on the nature of the remote functionality and the conditions of the preparation methods. The need for such protection is readily determined by one skilled in the art, and is described in examples carefully described in the above cited applications. The starting materials and reagents for the NRPA compounds employed in this invention are also readily available or can be easily synthesized by those skilled in the art using conventional methods of organic synthesis. Some of the compounds used herein are related to, or are derived from compounds found in nature and accordingly many such compounds are commercially available or are reported in the literature or are easily prepared from other commonly available substances by methods which are reported in the literature.

Some of the NRPA compounds employed in this invention are ionizable at physiological conditions. Thus, for example some of the compounds of this invention are acidic and they form a salt with a pharmaceutically acceptable cation. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, some of the NRPA compounds employed in this invention are basic, and they form a salt with a pharmaceutically acceptable anion. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, when the NRPA compounds employed in this invention form hydrates or solvates they are also within the scope of the invention.

Some of the compounds of this invention are chiral, and as such are subject to preparation via chiral synthetic routes, or separable by conventional resolution or chromatographic means. All optical forms of the compounds of this invention are within the scope of the invention.

The utility of the NRPA compounds employed in the present invention as medicinal agents in the treatment of pain in mammals (e.g. humans) is demonstrated by the activity of the compounds of this invention in conventional assays and, in particular the assays described below. These include neuronal nicotinic receptor binding and animal models of pain. Such assays also provide a means whereby the activities of the compounds of this invention can be compared between themselves and with the activities of other known compounds. The results of these comparisons are useful for determining dosage levels in mammals, including humans, for the treatment of such diseases.

Administration of the compositions of this invention can be via any method which delivers a compound of this invention systemically and/or locally. These methods include oral routes and transdermal routes, etc. Generally, the compounds of this invention are administered orally, but parenteral administration may be utilized (e.g., intravenous, intramuscular, subcutaneous or intramedullary). The two different compounds of this invention can be co-administered simultaneously or sequentially in any order, or a single pharmaceutical composition comprising a NRPA as described above and an analgesic agent as described above in a pharmaceutically acceptable carrier can be administered.

The amount and timing of compounds administered will, of course, be based on the judgement of the prescribing physician. Thus, because of patient to patient variability, the dosages given below are a guideline and the physician may titrate doses of the agent to achieve the activity that the physician considers appropriate for the individual patient. In considering the degree of activity desired, the physician must balance a variety of factors such as cognitive function, age of the patient, presence of preexisting disease, as well as presence of other diseases (e.g., cardiovascular). The following paragraphs provide preferred dosage ranges for the various components of this invention (based on average human weight of 70 kg).

### Biological Assays

### Procedures

Receptor binding assay: The effectiveness of the active compounds in suppressing nicotine binding to specific receptor sites is determined by the following procedure which is a modification of the methods of Lippiello, P. M. and Fernandes, K. G. (in The Binding of L-[³H]Nicotine To A Single Class of High-Affinity Sites in Rat Brain Membranes, Molecular Pharm., 29, 448-54, (1986)) and Anderson, D. J. and Arneric, S. P. (in Nicotinic Receptor Binding of ³H-Cystisine, ³H-Nicotine and ³H-Methylcarmbamylcholine In Rat Brain, European J. Pharm., 253, 261-67 (1994)). Male Sprague-Dawley rats (200-300 g) from Charles River were housed in groups in hanging stainless steel wire cages and were maintained on a 12 hour light/dark cycle (7 a.m.-7 p.m. light period). They received standard Purina Rat Chow and water ad libitum. The rats were killed by decapitation. Brains were removed immediately following decapitation. Membranes were prepared from brain tissue according to the methods of Lippiello and Fernandez (Molec Pharmacol, 29, 448-454, (1986) with some modifications. Whole brains were removed, rinsed with ice-cold buffer, and homogenized at 0° in 10 volumes of buffer (w/v) using a Brinkmann Polytron™, setting 6, for 30 seconds. The buffer consisted of 50 mM Tris HCl at a pH of 7.5 at room temperature. The homogenate was sedimented by centrifugation (10 minutes; 50,000×g; 0° to 4° C.). The supernatant was poured off and the membranes were gently resuspended with the Polytron and centrifuged again (10 minutes; 50,000×g; 0 to 4° C. After the second centrifugation, the membranes were resuspended in assay buffer at a concentration of 1.0 g/100 mL. The composition of the standard assay buffer was 50 mM Tris HCl, 120 mM NaCl, 5 mM KCl, 2 mM MgCl₂, 2 mM CaCl₂ and has a pH of 7.4 at room temperature.

Routine assays were performed in borosilicate glass test tubes. The assay mixture typically consisted of 0.9 mg of membrane protein in a final incubation volume of 1.0 mL. Three sets of tubes were prepared wherein the tubes in each set contained 50 μL of vehicle, blank, or test compound solution, respectively. To each tube was added 200 μL of [³H]-nicotine in assay buffer followed by 750 μL of the membrane suspension. The final concentration of nicotine in each tube was 0.9 nM. The final concentration of cytisine in the blank was 1 μM. The vehicle consisted of deionized water containing 30 μL of 1 N acetic acid per 50 mL of water. The test compounds and cytisine were dissolved in vehicle. Assays were initiated by vortexing after addition of the membrane suspension to the tube. The samples were incubated at 0° to 4° C. in an iced shaking water bath. Incubations were terminated by rapid filtration under vacuum through Whatman GF/B™ glass fiber filters using a Brandel™ multi-manifold tissue harvester. Following the initial filtration of the assay mixture, filters were washed two times with ice-cold assay buffer (5 m each). The filters were then placed in counting vials and mixed vigorously with 20 ml of Ready Safe™ (Beckman) before quantification of radioactivity. Samples were counted in a LKB Wallach Rackbeta™ liquid scintillation counter at 40-50% efficiency. All determinations were in triplicate.

Calculations: Specific binding (C) to the membrane is the difference between total binding in the samples containing vehicle only and membrane (A) and non-specific binding in the samples containing the membrane and cytisine (B), i.e.,

Specific binding=(C)=(A)−(B).

Specific binding in the presence of the test compound (E) is the difference between the total binding in the presence of the test compound (D) and non-specific binding (B), i.e., (E)=(D)−(B).

% Inhibition=(1−((E)/(C)) times 100.

The compounds of the invention that were tested in the above assay exhibited IC₅₀ values of less than 10 μM.

$$\frac{Assay methods for acute pain :}{Tail flick}$$

Tail-flick testing, which tests reflex nociceptive function, follows the procedure derived from D'Amour and Smith (D'Amour, F. E., and Smith, E., A method for determining loss of pain sensation, J. Pharmacol. Exp. Therapeutics, 72:74-79, 1941). The test is done with a standard apparatus obtained from Columbus instruments. A beam of radiant heat from a high intensity light is focussed on the tail while the animal is manually restrained. The response time is recorded, defined as the interval between the onset of the heat stimulus and the abrupt flick of the tail. As soon as the response occurs, the heat is removed from the tail. A cutoff time of 14 seconds (or less) is set to prevent damage to the tail of an animal with deficient sensory function. The test is administered to an animal three times in a session, varying the exact location of the heat spot on the tail to minimize sensitization and potential damage. Control animals have a tail flick response latency of approximately 4.5-5.0 seconds.

### Hot Plate

The hot-plate test, involving central as well as peripheral mechanisms of nociceptive responding, is conducted with an IITC model 39D Analgesia Meter. A rat is placed on a surface which is maintained at 55 degrees C. The surface is surrounded by a cylinder of clear plexiglass (10 in high). The latency between the time the rat is placed on the surface and the time it licks either hindpaw or attempts escape is the hot plate latency, and the animal is immediately removed from the apparatus at this time. One determination is recorded. To prevent tissue damage, tests of non-responsive animals are terminated after 40 sec., with that time assigned as the response latency. During the week prior to testing, the rats are given brief exposures to the non-functional hot-plate to adapt them to the testing situation. Control animals respond between 10-15 seconds.

$$\frac{Assay method for acute and chronic pain}{Formalin test}$$

This test does not allow escape from the stimulus, but is established as a standard means to test responses to a longer-duration nociceptive chemical stimulus. The response has two phases that appear to have separate mechanisms, distinct from one another and from the responses tested using the tests listed above, that can be independently investigated only by use of this test or similar tests (see (Tjolsen et al., 1992, cited below).

Animals are adapted to the testing situation without formalin injection during the week before the test. Fifty ml of 5% formalin solution is injected subcutaneously into the dorsal surface of the right hind paw with a 30 guage needle. The rat is then placed in an open plexiglass chamber to allow unhindered observation of the formalin-injected paw. Nociception-related behavior is quantified by counting the incidence of spontaneous flinching or shaking of the injected paw. Flinches are counted for each individual animal in periods of 1 minute starting at 1-2 min. after formalin injection, then at 5-minute intervals during the interval from 10-60 minutes. After the observation period of 1 hour, animals are sacrificed. Previous studies report that the duration of the painful stimulus in the formalin test is limited, and beyond one hour the pain is minimal (for review see Tjolsen, A., Berge, O -G., Hunskaar, S., Rosland, J. H., and Hole, K., The formalin test: an evaluation of the method, Pain, 51:5-17, 1992)

### Assay Method for Neuropathic Pain

Recently, several animal models of neuropathic pain have been developed in rats.

Bennett Model: [G. J. Bennett, Pain, 33, 87-107, 1988] Under anesthesia, the rat is placed in a prone position and an incision is made in the skin over the thigh. The fascia between the gluteus and biceps femoris muscle is dissected and the right common sciatic nerve is exposed at the level of the midthigh. Proximal to its trifurcation, the nerve is carefully dissected from its surrounding tissue over a distance of about 8 mm. In the experimental group, ligatures are loosely tied around the common sciatic nerve. A similar dissection is performed on the contralateral side, except that the nerve is not ligated (sham surgery). A group of control animals with bilateral sham surgery is also included. Comparison of the results of the experimental and control sides of the experimental rats allows the detection of possible contralateral effects of the nerve ligation.

In general, an effective dosage for the NRPA in the range of 0.001 to 200 mg/kg/day, preferably 0.001 to 10.0 mg/kg/day.

In particular, an effective dosage for propoxyphene, when used in the combination compositions and methods of this invention, is in the range of 0.1 to 5.7 mg/kg/day.

In particular, an effective dosage for meperidine, when used in the combination compositions and methods of this invention, is in the range of 0.1 to 2.0 mg/kg/day.

In particular, an effective dosage for hydromorphone, when used in the combination compositions and methods of this invention, is in the range of 0.01 to 0.2 mg/kg/day.

In particular, an effective dosage for hydrocodone, when used in the combination compositions and methods of this invention, is in the range of 0.04 to 0.6 mg/kg/day.

In particular, an effective dosage for morphine, when used in the combination compositions and methods of this invention, is in the range of 0.1 to 4.0 mg/kg/day.

In particular, an effective dosage for codeine, when used in the combination compositions and methods of this invention, is in the range of 0.01 to 0.3 mg/kg/day.

In particular, an effective dosage for 2-piperidinol-1-alkanol derivatives as described in U.S. Pat. No.5, 272,160, when used in the combination compositions and methods of this invention, is in the range of 0.1 to 20 mg/kg/day.

In particular, an effective dosage for eliprodil, when used in the combination compositions and methods of this invention, is in the range of 0.01 to 0.4 mg/kg/day

In particular, an effective dosage for ifenprodil, when used in the combination compositions and methods of this invention, is in the range of 0.01 to 0.3 mg/kg/day.

In particular, an effective dosage for rofecoxib, when used in the combination compositions and methods of this invention, is in the range of 0.1 to 0.35 mg/kg/day.

In particular, an effective dosage for celecoxib, when used in the combination compositions and methods of this invention, is in the range of 1.0 to 5.7 mg/kg/day.

In particular, an effective dosage for salycylic acid (aspirin), when used in the combination compositions and methods of this invention, is in the range of 1.0 to 50.0 mg/kg/day.

In particular, an effective dosage for diclofenac, when used in the combination compositions and methods of this invention, is in the range of 0.1 to 3.0 mg/kg/day.

In particular, an effective dosage for piroxicam, when used in the combination compositions and methods of this invention, is in the range of 0.1 to 0.3 mg/kg/day.

In particular, an effective dosage for indomethacin, when used in the combination compositions and methods of this invention, is in the range of 0.1 to 1.0 mg/kg/day.

In particular, an effective dosage for ibuprofen, when used in the combination compositions and methods of this invention, is in the range of 1.0 to 15.0 mg/kg/day.

In particular, an effective dosage for naproxen, when used in the combination compositions and methods of this invention, is in the range of 1.0 to 15.0 mg/kg/day.

In particular, an effective dosage for gabapentin, when used in the combination compositions and methods of this invention, is in the range of 10.0 to 35.0 mg/kg/day.

In particular, an effective dosage for carbemazepine, when used in the combination compositions and methods of this invention, is in the range of 1.0 to 20.0 mg/kg/day.

In particular, an effective dosage for pregabalin, when used in the combination compositions and methods of this invention, is in the range of 1.0 to 10.0 mg/kg/day.

In particular, an effective dosage for topiramate, when used in the combination compositions and methods of this invention, is in the range of 0.1 to 6.0 mg/kg/day.

In particular, an effective dosage for valproic acid, when used in the combination compositions and methods of this invention, is in the range of 1.0 to 60 mg/kg/day.

In particular, an effective dosage for sumatriptan, when used in the combination compositions and methods of this invention, is in the range of 0.1 to 1.5 mg/kg/day.

In particular, an effective dosage for elitriptan, when used in the combination compositions and methods of this invention, is in the range of 0.1 to 1.1 mg/kg/day.

In particular, an effective dosage for rizatriptan, when used in the combination compositions and methods of this invention, is in the range of 0.05 to 0.15 mg/kg/day.

In particular, an effective dosage for zolmitriptan, when used in the combination compositions and methods of this invention, is in the range of 0.01 to 0.1 mg/kg/day.

In particular, an effective dosage for naratriptan, when used in the combination compositions and methods of this invention, is in the range of 0.01 to 0.07 mg/kg/day.

In particular, an effective dosage for flexeril, when used in the combination compositions and methods of this invention, is in the range of 0.1 to 0.9 mg/kg/day.

In particular, an effective dosage for carisoprodol, when used in the combination compositions and methods of this invention, is in the range of 1.0 to 20.0 mg/kg/day.

In particular, an effective dosage for robaxisal, when used in the combination compositions and methods of this invention, is in the range of 1.0 to 70.0 mg/kg/day.

In particular, an effective dosage for norgesic, when used in the combination compositions and methods of this invention, is in the range of 0.1 to 1.5 mg/kg/day.

In particular, an effective dosage for dantrium, when used in the combination compositions and methods of this invention, is in the range of 0.1 to 1.0 mg/kg/day.

In particular, an effective dosage for diazepam, when used in the combination compositions and methods of this invention, is in the range of 0.01 to 0.5 mg/kg/day.

In particular, an effective dosage for chlordiazepoxide, when used in the combination compositions and methods of this invention, is in the range of 0.05 to 1.4 mg/kg/day.

In particular, an effective dosage for alprazolam, when used in the combination compositions and methods of this invention, is in the range of 0.001 to 0.05 mg/kg/day.

In particular, an effective dosage for lorazepam, when used in the combination compositions and methods of this invention, is in the range of 0.005 to 0.15 mg/kg/day.

In particular, an effective dosage for acetominophen, when used in the combination compositions and methods of this invention, is in the range of 1.0 to 5.0 mg/kg/day.

In particular, an effective dosage for nitrous oxide, when used in the combination compositions and methods of this invention, is in the range of 10% to 50% mg/kg/day.

In particular, an effective dosage for halothane, when used in the combination compositions and methods of this invention, is in the range of 0.1% to 3.0%.

In particular, an effective dosage for lidocaine, when used in the combination compositions and methods of this invention, is in the range of 0.1% to 2.0%

In particular, an effective dosage for etidocaine, when used in the combination compositions and methods of this invention, is in the range of 0.1% to 1.5%

In particular, an effective dosage for ropivacaine, when used in the combination compositions and methods of this invention, is in the range of 0.1% to 1.0%

In particular, an effective dosage for chloroprocaine, when used in the combination compositions and methods of this invention, is in the range of 0.1% to 2.0% mg/kg/day.

In particular, an effective dosage for sarapin, when used in the combination compositions and methods of this invention, is in the range of 0.1 to 10 mls of a sterile aqueous solution of soluble salts of the volatile bases from Sarraceniaceae (Pitcher Plant).

In particular, an effective dosage for bupivacaine, when used in the combination compositions and methods of this invention, is in the range of 0.1% to 0.75%

In particular, an effective dosage for capsaicin receptor agonists such as Arthricare, when used in the combination compositions and methods of this invention, is in the range of 0.01% to 0.1%

In particular, an effective dosage for desipramine, when used in the combination compositions and methods of this invention, is in the range of 0.1 to 3.0 mg/kg/day.

In particular, an effective dosage for amitriptyline, when used in the combination compositions and methods of this invention, is in the range of 0.1 to 2.0 mg/kg/day.

In particular, an effective dosage for doxepin, when used in the combination compositions and methods of this invention, is in the range of 0.1 to 2.0 mg/kg/day.

In particular, an effective dosage for perphenazine, when used in the combination compositions and methods of this invention, is in the range of 0.01 to 0.2 mg/kg/day.

In particular, an effective dosage for protriptyline, when used in the combination compositions and methods of this inventon, is in the range of 0.05 to 0.9 mg/kg/day.

In particular, an effective dosage for tranylcypromine, when used in the combination compositions and methods of this invention, is in the range of 0.1 to 0.9 mg/kg/day.

In particular, an effective dosage for baclofen, when used in the combination compositions and methods of this invention, is in the range of 0.1 to 0.5 mg/kg/day.

In particular, an effective dosage for clonidine, when used in the combination compositions and methods of this invention, is in the range of 0.001 to 0.03 mg/kg/day.

In particular, an effective dosage for mexelitine, when used in the combination compositions and methods of this invention, is in the range of 1.0 to 15.0 mg/kg/day.

In particular, an effective dosage for diphenhydramine, when used in the combination compositions and methods of this invention, is in the range of 0.1 to 4.0 mg/kg/day.

In particular, an effective dosage for hydroxyzine, when used in the combination compositions and methods of this invention, is in the range of 0.1 to 5.0 mg/kg/day.

In particular, an effective dosage for caffeine, when used in the combination compositions and methods of this invention, is in the range of 0.1 to 15.0 mg/kg/day.

In particular, an effective dosage for prednisone, when used in the combination compositions and methods of this invention, is in the range of 0.01 to 1.0 mg/kg/day.

In particular, an effective dosage for methyl-predinsone, when used in the combination compositions and methods of this invention, is in the range of 0.01 to 0.5 mg/kg/day.

In particular, an effective dosage for decadron, when used in the combination compositions and methods of this invention, is in the range of 0.005 to 0.1 mg/kg/day.

In particular, an effective dosage for sertraline, when used in the combination compositions and methods of this invention, is in the range of 0.1 to 3.0 mg/kg/day.

In particular, an effective dosage for paroxetine, when used in the combination compositions and methods of this invention, is in the range of 0.1 to 0.7 mg/kg/day.

In particular, an effective dosage for fluoxetine when used in combination composition and methods of this invention, is in the range of 0.1 to 1.0 mg/kg/day.

In particular, an effective dosage for tramodol, when used in the combination compositions and methods of this invention, is in the range of 0.5 to 5.0 mg/kg/day.

In particular, an effective dosage for levodopa, when used in the combination compositions and methods of this invention, is in the range of 1.0 to 15.0 mg/kg/day.

In particular, an effective dosage for dextromethorphan, when used in the combination compositions and methods of this invention, is in the range of 0.1 to 1.5 mg/kg/day.

In particular, an effective dosage for substance P antagonists, when used in the combination compositions and methods of this invention, is in the range of 0.01 to 15.0 mg/kg/day.

In particular, an effective dosage for Ziconotide®, when used in combination compositions and methods of this invention, is in the range of 0.1 to 1.0 mg/kg/day.

In particular, an effective dosage for botulinum toxin, when used in the combination compositions and methods of this invention, is in the range of 1 to 10 units/day.

The compositions of the present invention are generally administered in the form of a pharmaceutical composition comprising at least one of the compounds of this invention together with a pharmaceutically acceptable vehicle or diluent. Thus, the compounds of this invention can be administered individually or together in any conventional oral, parenteral or transdermal dosage form.

For oral administration a pharmaceutical composition can take the form of solutions, suspensions, tablets, pills, capsules, powders, and the like. Tablets containing various excipient such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g.,topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the pain of the subject being treated.

1. A pharmaceutical composition for the treatment of acute, chronic and/or neuropathic pain and migraine comprising (a) a nicotine receptor partial agonist or a pharmaceutically acceptable salt thereof; (b) an analgesic agent or pharmaceutically acceptable salt thereof and (c) a pharmaceutically acceptable carrier; wherein the active agents “a” and “b” above are present in amounts that render the composition effective in treating acute, chronic and/or neuropathic pain, and migraine.

2. The pharmaceutical composition according to claim 1, wherein said analgesic agent is selected from opioid analgesics, NMDA antagonists, substance P antagonists, COX 1 and COX 2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, N-type calcium channel antagonists, and botulinum toxin.

3. The pharmaceutical composition according to claim 2, wherein said opioid analgesic agent is selected from propoxyphene (Darvon), meperidine (Demerol), hydromorphone (Dilaudid), hydrocodone (Lortab), morphine, codeine and tramodol; their pharmaceutically active salts and their optical isomers.

4. The pharmaceutical composition according to claim 2 wherein said NMDA antagonist analgesic agent is selected from 2-piperdino-1 alkanol derivates, dextromethorphan, eliprodil, and ifenprodil, their pharmaceutically active salts and their optical isomers.

5. The pharmaceutical composition according to claim 2, wherein the substance P antagonist analgesic agent is selected from
(6-Methoxy-3-trifluoromethyl-benzo[d]isoxazol-5-ylmethyl)-(2-phenyl-piperidin-3-yl)-amine;
6-Methoxy-1-methyl-7-[(2-phenyl-1-propyl-piperidin-3-ylamino)-methyl]-3,4-dihydro-1H-quinolin-2-one;
6-Methoxy-1-methyl-7-{[1-(5-oxo-2,5-dihydro-1H-[1,2,4]triazol-3-ylmethyl) piperidin-3-ylamino]-methyl}-3,4-dihydro-1H-quinolin-2-one;
3-(2-Methoxy-5-trifluoromethoxy-phenyl)-6-phenyl-1,7-diaza-spiro[4.5]decane;
6-Methoxy-1-methyl-7-[(2-phenyl-piperidin-3-ylamino)-methyl]-3,4-dihydro-1H-quinolin-2-one;
[2-Methoxy-5-(2,2,2-trifluoro-1-trifluoromethyl-ethyl)-benzyl]-(2-phenyl-piperidin-3-yl)-amine;
[5-(1,1-Dimethyl-prop-2-ynyl)-2-methoxy-benzyl]-(2-phenyl-piperidin-3-yl)-amine;
7-Methoxy-1-methyl-6-[(2-phenyl-piperidin-3-ylamino)-methyl]-3,4-dihydro-1H-quinolin-2-one;
[2-Methoxy-5-(2,2,2-trifluoro-1,1-dimethyl-ethyl)-benzyl]-(2-phenyl-piperidin-3-yl)-amine;
(7-Methoxy-4-methyl-3,4-dihydro-2H-benzo[1,4]oxazin-6-ylmethyl)-(2-phenyl-piperidin-3-yl)-amine;
[2-Methoxy-5-(1-methyl-1-trifluoromethyl-prop-2-ynyl)-benzyl]-(2-phenyl-piperidin-3-yl)-amine;
(6-Methoxy-1-methyl-1-trifluoromethyl-isochroman-7-ylmethyl)-(2-phenyl-piperidin-3-yl)-amine;
2-{3-[(2-Benzhydryl-1-aza-bicyclo[2.2.2]oct-3-ylamino)-methyl]-4-methoxy-phenyl }-2-methyl-propan-1-ol;
(2S,3S)-N-[(5-oxo-1H,4H-1,2,4-triazolo)methyl]-2-(4-fluorophenyl)-3-(3,5-ditrifluoromethyl)benzyloxymorpholine;
3-(3,5-Bis-trifluoromethyl-benzyloxy)-2-phenyl-piperidine;
5-[2-(3,5-Bis-trifluoromethyl-benzyloxy)-3-phenyl-morpholin-4-ylmethyl]-2,4-dihydro-[1,2,4]triazol-3-one;
(2S,3S)-3-(2-Methoxy-5-(trifluoromethoxy)benzyl)amino-2-phenylpiperidine;
(2S,3S)-N-(5-isopropyl-2-methoxyphenyl)methyl-2-diphenylmethyl-1-azabicyclo[2.2.2]-octan-3-amine;
(2S,3S)-N-(5-tert-butyl-2-methoxyphenyl)methyl-2-diphenylmethyl-1-azabicyclo[2.2.2]-octane-3-amine;
(2S,3S)-N-(5-ethyl-2-methoxyphenyl)methyl-2-diphenylmethyl-1-azabicyclo[2.2.2]-octan-3-amine; and
(2S,3S)-N-(5-n-propyl-2-methoxyphenyl)methyl-2-diphenylmethyl-1-azabicyclo[2.2.2]-octane-3-amine, their pharmaceutically active salts and their optical isomers.

6. The pharmaceutical composition according to claim 2 wherein the COX 2 inhibitor analgesic agent is selected from rofecoxib and celecoxib their pharmaceutically active salts and their optical isomers.

7. The pharmaceutical composition according to claim 2 wherein the anesthetic analgesic agent agent is selected from nitrous oxide, halothane, lidocaine, etidocaine, ropivacaine, chloroprocaine, sarapin and bupivacaine their pharmaceutically active salts and their optical isomers.

8. The pharmaceutical composition according to claim 2 wherein the benzodiazepine analgesic agent is selected from diazepam, chlordiazepoxide, alprazolam, and lorazepam their pharmaceutically active salts and their optical isomers.

9. The pharmaceutical composition according to claim 2 wherein the skeletal muscle relaxant analgesic agent is selected from flexeril, carisoprodol, robaxisal, norgesic and dantrium their pharmaceutically active salts and their optical isomers.

10. The pharmaceutical composition according to claim 2 wherein the migraine therapeutic agent is selected from elitriptan, sumatriptan, rizatriptan, zolmitriptan, and naratriptan their pharmaceutically active salts and their optical isomers.

11. The pharmaceutical composition according to claim 2 wherein the anticonvulsant analgesic agent is selected from gabapentin, pregabalin, carbamazepine, and topiramate and valproic acid their pharmaceutically active salts and their optical isomers.

12. The pharmaceutical composition according to claim 2 wherein the COX 1 inhibitor analgesic agent is selected from salycylic acid, acetominophen, diclofenac, piroxican indomethacin, ibuprofen, and naproxen their pharmaceutically active salts and their optical isomers.

13. The pharmaceutical composition according to claim 2 wherein the tricyclic antidepressant analgesic agent is selected from amitriptyline, desipramine, perphenazine, protriptyline, and tranylcypromine their pharmaceutically active salts and their optical isomers.

14. The pharmaceutical composition according to claim 1 wherein the analgesic agent is chosen from baclofen, clonidine, mexilitene, diphenyl-hydramine, hydroxysine, caffeine, prednisone, methylprednisone, decadron, paroxetine, sertraline, fluoxetine, tramodol, Ziconotide® and levodopa their pharmaceutically active salts and their optical isomers.

15. The pharmaceutically composition according to claim 1, wherein said nicotine receptor partial agonist is selected from
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-ethyl-1,2,3,4,5,6-hexahydro-1,5-methano-pyrido[1,2-a][1,5]diazocin-8-one;
9-methyl-1,2,3,4,5,6-hexahydro-1,5-methano-pyrido[1,2-a][1,5]diazocin-8-one;
9-phenyl-1,2,3,4,5,6-hexahydro-1,5-methano-pyrido[1,2-a][1,5]diazocin-8-one;
9-vinyl-1,2,3,4,5,6-hexahydro-1,5-methano-pyrido[1,2-a][1,5]diazocin-8-one;
9-bromo-3-methyl-1,2,3,4,5,6-hexahydro-1,5-methano-pyrido[1,2-a][1,5]diazocin-8-one;
3-benzyl-9-bromo-1,2,3,4,5,6-hexahydro-1,5-methano-pyrido[1,2-a][1,5]diazocin-8-one;
3-benzyl-9-chloro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-ethynyl-1,2,3,4,5,6-hexahydro-1,5-methano-pyrido[1,2a][1,5]diazocin-8-one;
9-(2-propenyl)-1,2,3,4,5,6-hexahydro-1,5-methano-pyrido[1,2a][1,5]diazocin-8-one;
9-(2-propyl)-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
9-(4-fluorophenyl)-1,2,3,4,5,6-hexahydro-1,5-methano-pyrido[1,2a][1,5]diazocin-8-one;
9-(3-fluorophenyl)-1,2,3,4,5,6-hexahydro-1,5-methano-pyrido[1,2a][1,5]diazocin-8-one;
9-(3,5-difluorophenyl)-1,2,3,4,5,6-hexahydro-1,5-methano-pyrido[1,2a][1,5]diazocin-8-one;
9-(2,4-difluorophenyl)-1,2,3,4,5,6-hexahydro-1,5-methano-pyrido[1,2a][1,5]diazocin-8-one;
9-(2,5-difluorophenyl)-1,2,3,4,5,6-hexahydro-1,5-methano-pyrido[1,2a][1,5]diazocin-8-one;
6-methyl-5-oxo-6,13-diazatetracyclo[9.3.1.0^{2,10}.0^{4,8}]pentadeca-2(10),3,8-triene;
5-oxo-6,13-diazatetracyclo[9.3.1.0^{2,10}.0^{4,8}]pentadeca-2(10),3,8-triene;
6-oxo-5,7,13-triazatetracyclo[9.3.1.0^{2,10}.0^{4,8}]pentadeca-2(10),3,8-triene;
4,5-difluoro-10-aza-tricyclo[6.3.1.0^{2,7}]dodeca-2(7),3,5-triene;
5-fluoro-10-aza-tricyclo[6.3.1.0^{2,7}]dodeca-2(7),3,5-triene-4-carbonitrile;
4-ethynyl-5-fluoro-10-aza-tricyclo[6.3.1.0^{2,7}]dodeca-2(7),3,5-triene;
5-ethynyl-10-aza-tricyclo[6.3.1.0^{2,7}]dodeca-2(7),3,5-triene-4-carbonitrile;
6-methyl-5-thia-5-dioxa-6,13-diazatetracyclo[9.3.1.0^{2,10}.0^{4,8}]pentadeca-2(10),3,8-triene;
10-aza-tricyclo[6.3.1.0^{2,7}]dodeca-2(7),3,5-triene;
4-fluoro-10-aza-tricyclo[6.3.1.0^{2,7}]dodeca-2(7),3,5-triene;
4-methyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
7-methyl-5,7,13-triazatetracyclo[9.3.1.0^{2,10}.0^{4,8}]pentadeca-2(10),3,5,8-tetraene;
6-methyl-5,7,13-triazatetracyclo[9.3.1.0^{2,10}.0^{4,8}]pentadeca-2(10),3,5,8-tetraene;
6,7-dimethyl-5,7,13-triazatetracyclo[9.3.1.0^{2,10}.0^{4,8}]pentadeca-2(10),3,5,8-tetraene;
6-methyl-7-phenyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
14-methyl-5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
4-chloro-10-azatricyclo[6.3.1.0^{2,7}]dodeca-2(7),3,5-triene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
10-azatricyclo[6.3.1.0^{2,7}]dodeca-2(7),3,5-trien-4-ol;
7-methyl-5-oxa-6,13-diazatetracyclo[9.3.1.0^{2,10}.0^{4,8}]pentadeca-2,4(8),6,9-tetraene;
4,5-dichloro-10-azatricyclo[6.3.1.0^{2,7}]dodeca-2(7),3,5-triene;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
5,7,14-triazatetracyclo[10.3.1.0^{2,10}.0^{4,8}]hexadeca-2(10),3,5,8-tetraene;
5,6-dimethyl-5,7,14-triazatetracyclo[10.3.1.0^{2,10}.0^{4,8}]hexadeca-2(10),3,6,8-tetraene;
5-methyl-5,7,14-triazatetracyclo[10.3.1.0^{2,10}.0^{4,8}]hexadeca-2(10),3,6,8-tetraene;
6-(trifluoromethyl)-7-thia-5,14-diazatetracyclo[10.3.1.0^{2,10}.0⁴ 8]hexadeca-2(10),3,5,8-tetraene;
5,8,15-triazatetracyclo[11.3.1.0^{2,11}.0^{4,9}]heptadeca-2(11),3,5,7,9-pentaene;
7-methyl-5,8,15-triazatetracyclo[11.3.1.0^{2,11}.0^{4,9}]heptadeca-2(11),3,5,7,9-pentaene;
6-methyl-5,8,15-triazatetracyclo[11.3.1.0^{2,11}.0^{4,9}]heptadeca-2(11),3,5,7,9-pentaene;
6,7-dimethyl-5,8,15-triazatetracyclo[11.3.1.0^{2,11}.0^{4,9}]heptadeca-2(11),3,5,7,9-pentaene;
7-oxa-5,14-diazatetracyclo[10.3.1.0^{2,10}.0^{4,8}]hexadeca-2(0),3,5,8-tetraene;
6-methyl-7-oxa-5,14-diazatetracyclo[10.3.1.0^{2,10}.0^{4,8}]hexadeca-2(10),3,5,8-tetraene;
5-methyl-7-oxa-6,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
7-methyl-5-oxa-6,14-diazatetracyclo[10.3.1.0^{2,10}.0^{4,8}]hexadeca-2(10),3,6,8-tetraene;
4,5-difluoro-11-azatricyclo[7.3.1.0^{2,7}]trideca-2(7),3,5-triene;
4-chloro-5-fluoro-11-azatricyclo[7.3.1.0^{2,7}]trideca-2(7),3,5-triene;
5-chloro-4-fluoro-11-azatricyclo[7.3.1.0^{2,7}]trideca-2(7),3,5-triene;
4-(1-ethynyl)-5-fluoro-11-azatricyclo[7.3.1.0^{2,7}]trideca-2(7),3,5-triene;
5-(1-ethynyl)-4-fluoro-11-azatricyclo[7.3.1.0^{2,7}]trideca-2(7),3,5-triene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
11-aza-tricyclo[7.3.1.0^{2,7}]trideca-2(7),3,5-trien-6-ol;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol;
4-nitro-11-aza-tricyclo[7.3.1.0^{2,7}]trideca-2(7),3,5-triene;
5-nitro-11-aza-tricyclo[7.3.1.0^{2,7}]trideca-2(7),3,5-triene;
5-fluoro-11-aza-tricyclo[7.3.1.0^{2,7}]trideca-2(7),3,5-triene;
6-hydroxy-5-methoxy-11-aza-tricyclo[7.3.1.0^{2,7}]trideca-2(7),3,5-triene; and their pharmaceutically acceptable salts and their optical isomers.

16. The pharmaceutical composition according to claim 16 wherein said nicotine receptor partial agonist is selected from
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
6-methyl-5-thia-5-dioxa-6,13-diazatetracyclo[9.3.1.0^{2,10}.0^{4,8}]pentadeca-2(10),3,8-triene;
4-fluoro-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
6-methyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,10}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol, and their pharmaceutically acceptable salts and their optical isomers thereof.

17. A method of treating acute, chronic and/or neuropathic pain and migraine in a mammal comprising administering to said mammal, respectively a pain attenuating effective amount of a pharmaceutical composition comprising: (a) a nicotine receptor partial agonist or a pharmaceutically acceptable salt thereof; (b) an analgesic agent or pharmaceutically acceptable salt thereof and (c) a pharmaceutically acceptable carries, wherein the active agents “a” and “b” above are present in amounts that render the composition effective in treating acute, chronic and/or neuropathic pain and migraine.

18. The method according to claim 17 wherein the analgesics are selected from opioid analgesics, NMDA antagonists, substance P antagonists, COX 1 and COX 2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, N-type calcium channel antoginists and botulinum toxin or their pharmaceutically acceptable salt or optical isomers.

19. The method according to claim 18 wherein said NMDA antagonist analgesic agent is selected from 2-piperidinol-1 alkanol derivatives, dextromethorphan, eliprodil, and ifenprodil, their pharmaceutically active salts and their optical isomers.

20. The method according to claim 18 wherein the substance P antagonists are selected from
(6-Methoxy-3-trifluoromethyl-benzo[d] isoxazol-5-ylmethyl)-(2-phenyl-piperidin-3-yl)-amine;
6-Methoxy-1-methyl-7-[(2-phenyl-1-propyl-piperidin-3-ylamino)-methyl]-3,4-dihydro-1H-quinolin-2-one;
6-Methoxy-1-methyl-7-{[1-(5-oxo-2,5-dihydro-1H-[1,2,4]triazol-3-ylmethyl)-2-phenyl-piperidin-3-ylamino]-methyl}-3,4-dihydro-1H-quinolin-2-one;
3-(2-Methoxy-5-trifluoromethoxy-phenyl)-6-phenyl-1,7-diaza-spiro[4.5]decane;
6-Methoxy-1-methyl-7-[(2-phenyl-piperidin-3-ylamino)-methyl]-3,4-dihydro-1H-quinolin-2-one;
[2-Methoxy-5-(2,2,2-trifluoro-1-trifluoromethyl-ethyl)-benzyl]-(2-phenyl-piperidin-3-yl)-amine;
[5-(11-Dimethyl-prop-2-ynyl)-2-methoxy-benzyl]-(2-phenyl-piperidin-3-yl)-amine;
7-Methoxy-1-methyl-6-[(2-phenyl-piperidin-3-ylamino)-methyl]-3,4-dihydro-1H-quinolin-2-one;
[2-Methoxy-5-(2,2,2-trifluoro-1,1-dimethyl-ethyl)-benzyl]-(2-phenyl-piperidin-3-yl)-amine;
(7-Methoxy-4-methyl-3,4-dihydro-2H-benzo[1,4]oxazin-6-ylmethyl)-(2-phenyl-piperidin-3-yl)-amine;
[2-Methoxy-5-(1-methyl-1-trifluoromethyl-prop-2-ynyl)-benzyl]-(2-phenyl-piperidin-3-yl)-amine;
(6-Methoxy-1-methyl-1-trifluoromethyl-isochroman-7-ylmethyl)-(2-phenyl-piperidin-3-yl)-amine; 2-{3-[(2-Benzhydryl-1-aza-bicyclo[2.2.2]oct-3-ylamino)-methyl]-4-methoxy-phenyl}-2-methyl-propan-1-ol;
(2S,3S)-N-[(5-oxo-1H,4H-1,2,4-triazolo)methyl]-2-(4-fluorophenyl)-3-(3,5-ditrifluoromethyl)benzyloxymorpholine;
3-(3,5-Bis-trifluoromethyl-benzyloxy)-2-phenyl-piperidine;
5-[2-(3,5-Bis-trifluoromethyl-benzyloxy)-3-phenyl-morpholin-4-ylmethyl]-2,4-dihydro-[1,2,4]triazol-3-one;
(2S,3S)-3-(2-Methoxy-5-(trifluoromethoxy)benzyl)amino-2-phenylpiperidine;
(2S,3S)-N-(5-isopropyl-2-methoxyphenyl)methyl-2-diphenylmethyl-1-azabicyclo[2.2.2]-octan-3-amine;
(2S,3S)-N-(5-tert-butyl-2-methoxyphenyl)methyl-2-diphenylmethyl-1-azabicyclo[2.2.2]-octane-3-amine;
(2S,3S)-N-(5-ethyl-2-methoxyphenyl)methyl-2-diphenylmethyl-1-azabicyclo[2.2.2]-octan-3-amine; and
(2S,3S)-N-(5-n-propyl-2-methoxyphenyl)methyl-2-diphenylmethyl-1-azabicyclo[2.2.2]-octane-3-amine or a pharmaceutically acceptable salt or an optical isomer thereof.

21. The method according to claim 18 wherein the COX 2 inhibitor analgesic agent is selected from rofecoxib and celecoxib their pharmaceutically active salts and their optical isomers.

22. The method according to claim 18 wherein the anesthetic analgesic agent agent is selected from nitrous oxide, halothane, lidocaine, etidocaine, ropivacaine, chloroprocaine, sarapin and bupivacaine their pharmaceutically active salts and their optical isomers.

23. The method according to claim 18 wherein the benzodiazepine analgesic agent is selected from diazepam, chlordiazepoxide, alprazolam, and lorazepam their pharmaceutically active salts and their optical isomers.

24. The method according to claim 18 wherein the skeletal muscle relaxant analgesic agent is selected from flexeril, carisoprodol, robaxisal, norgesic and dantrium their pharmaceutically active salts and their optical isomers.

25. The method according to claim 18 wherein the migraine therapeutic agent is selected from elitriptan, sumatriptan, rizatriptan, zolmitriptan, and naratriptan their pharmaceutically active salts and their optical isomers.

26. The method according to claim 18 wherein the anticonvulsant analgesic agent is selected from gabapentin, pregabalin, carbamazepine, and topiramate and valproic acid their pharmaceutically active salts and their optical isomers.

27. The method according to claim 18 wherein the COX 1 inhibitor analgesic agent is selected from salycylic acid, acetominophen, diclofenac, piroxican indomethacin, ibuprofen, and naproxen their pharmaceutically active salts and their optical isomers.

28. The method according to claim 18 wherein the tricyclic antidepressant analgesic agent is selected from amitriptyline, desipramine, perphenazine, protriptyline, and tranylcypromine their pharmaceutically active salts and their optical isomers.

29. The method according to claim 18 wherein the analgesic agent is chosen from baclofen, clonidine, mexilitene, diphenyl-hydramine, hydroxysine, caffeine, prednisone, methylprednisone, decadron, paroxetine, sertraline, fluoxetine, tramodol, Ziconotide® and levodopa their pharmaceutically active salts and their optical isomers.

30. The method according to claim 17, wherein the nicotine partial agonist is selected from
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-ethyl-1,2,3,4,5,6-hexahydro-1,5-methano-pyrido[1,2-a][1,5]diazocin-8-one;
9-methyl-1,2,3,4,5,6-hexahydro-1,5-methano-pyrido[1,2-a][1,5]diazocin-8-one;
9-phenyl-1,2,3,4,5,6-hexahydro-1,5-methano-pyrido[1,2-a][1,5]diazocin-8-one;
9-vinyl-1,2,3,4,5,6-hexahydro-1,5-methano-pyrido[1,2-a][1,5]diazocin-8-one;
9-bromo-3-methyl-1,2,3,4,5,6-hexahydro-1,5-methano-pyrido[1,2-a][1,5]diazocin-8-one;
3-benzyl-9-bromo-1,2,3,4,5,6-hexahydro-1,5-methano-pyrido[1,2-a][1,5]diazocin-8-one;
3-benzyl-9-chloro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-ethynyl-1,2,3,4,5,6-hexahydro-1,5-methano-pyrido[1,2a][1,5]diazocin-8-one;
9-(2-propenyl)-1,2,3,4,5,6-hexahydro-1,5-methano-pyrido[1,2a][1,5]diazocin-8-one;
9-(2-propyl)-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
9-(4-fluorophenyl)-1,2,3,4,5,6-hexahydro-1,5-methano-pyrido[1,2a][1,5]diazocin-8-one;
9-(3-fluorophenyl)-1,2,3,4,5,6-hexahydro-1,5-methano-pyrido[1,2a][1,5]diazocin-8-one;
9-(3,5-difluorophenyl)-1,2,3,4,5,6-hexahydro-1,5-methano-pyrido[1,2a][1,5]diazocin-8-one;
9-(2,4-difluorophenyl)-1,2,3,4,5,6-hexahydro-1,5-methano-pyrido[1,2a][1,5]diazocin-8-one;
9-(2,5-difluorophenyl)-1,2,3,4,5,6-hexahydro-1,5-methano-pyrido[1,2a][1,5]diazocin-8-one;
6-methyl-5-oxo-6,13-diazatetracyclo[9.3.1.0^{2,10}.0^{4,8}]pentadeca-2(10),3,8-triene;
5-oxo-6,13-diazatetracyclo[9.3.1.0^{2,10}.0^{4,8}]pentadeca-2(10),3,8-triene;
6-oxo-5,7,13-triazatetracyclo[9.3.1.0^{2,10}.0^{4,8}]pentadeca-2(10),3,8-triene;
4,5-difluoro-10-aza-tricyclo[6.3.1.0^{2,7}]dodeca-2(7),3,5-triene;
5-fluoro-10-aza-tricyclo[6.3.1.0^{2,7}]dodeca-2(7),3,5-triene-4-carbonitrile;
4-ethynyl-5-fluoro-10-aza-tricyclo[6.3.1.0^{2,7}]dodeca-2(7),3,5-triene;
5-ethynyl-10-aza-tricyclo[6.3.1.0^{2,7}]dodeca-2(7),3,5-triene-4-carbonitrile;
6-methyl-5-thia-5-dioxa-6,13-diazatetracyclo[9.3.1.0^{2,10}.0^{4,8}]pentadeca-2(10),3,8-triene;
10-aza-tricyclo[6.3.1.0^{2,7}]dodeca-2(7),3,5-triene;
4-fluoro-10-aza-tricyclo[6.3.1.0^{2,7}]dodeca-2(7),3,5-triene;
4-methyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
7-methyl-5,7,13-triazatetracyclo[9.3.1.0^{2,10}.0^{4,8}]pentadeca-2(10),3,5,8-tetraene;
6-methyl-5,7,13-triazatetracyclo[9.3.1.0^{2,10}.0^{4,8}1]pentadeca-2(10),3,5,8-tetraene;
6,7-dimethyl-5,7,13-triazatetracyclo[9.3.1.0^{2,10}.0^{4,8}]pentadeca-2(10),3,5,8-tetraene;
6-methyl-7-phenyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
14-methyl-5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
4-chloro-10-azatricyclo[6.3.1.0^{2,7}]dodeca-2(7),3,5-triene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
10-azatricyclo[6.3.1.0^{2,7}]dodeca-2(7),3,5-trien-4-ol;
7-methyl-5-oxa-6,13-diazatetracyclo[9.3.1.0^{2,10}.0^{4,8}]pentadeca-2,4(8),6,9-tetraene;
4,5-dichloro-10-azatricyclo[6.3.1.0^{2,7}]dodeca-2(7),3,5-triene;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-l -propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
5,7,14-triazatetracyclo[10.3.1.0^{2,10}.0^{4,8}]hexadeca-2(10),3,5,8-tetraene;
5,6-dimethyl-5,7,14-triazatetracyclo[10.3.1.0^{2,10}.0^{4,8}]hexadeca-2(10),3,6,8-tetraene;
5-methyl-5,7,14-triazatetracyclo[10.3.1.0^{2,10}.0^{4,8}]hexadeca-2(10),3,6,8-tetraene;
6-(trifluoromethyl)-7-thia-5,14-diazatetracyclo[10.3.1.0^{2,10}.0^{4,8}]hexadeca-2(10),3,5,8-tetraene;
5,8,15-triazatetracyclo[11.3.1.0^{2,11}.0^{4,9}]heptadeca-2(11),3,5,7,9-pentaene;
7-methyl-5,8,15-triazatetracyclo[11.3.1.0^{2,11}.0^{4,9}]heptadeca-2(11),3,5,7,9-pentaene;
6-methyl-5,8,15-triazatetracyclo[11.3.1.0^{2,11}.0^{4,9}]heptadeca-2(11),3,5,7,9-pentaene;
6,7-dimethyl-5,8,15-triazatetracyclo[11.3.1.0^{2,11}.0^{4,9}]heptadeca-2(11),3,5,7,9-pentaene;
7-oxa-5,14-diazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
5-methyl-7-oxa-6,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
7-methyl-5-oxa-6,14-diazatetracyclo[10.3.1.0^{2,10}.0^{4,8}]hexadeca-2(10),3,6,8-tetraene;
4,5-difluoro-11-azatricyclo[7.3.1.0^{2,7}]trideca-2(7),3,5-triene;
4-chloro-5-fluoro-11-azatricyclo[7.3.1.0^{2,7}]trideca-2(7),3,5 -triene;
5-chloro-4-fluoro-11-azatricyclo[7.3.1.0^{2,7}]trideca-2(7),3,5-triene;
4-(-ethynyl)-5-fluoro-11-azatricyclo[7.3.1.0^{2,7}]trideca-2(7),3,5-triene;
5-(1-ethynyl)-4-fluoro-11-azatricyclo[7.3.1.0^{2,7}]trideca-2(7),3,5-triene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
11-aza-tricyclo[7.3.1.0^{2,7}]trideca-2(7),3,5-trien-6-ol;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol;
4-nitro-11-aza-tricyclo[7.3.1.0^{2,7}]trideca-2(7),3,5-triene;
5-nitro-11-aza-tricyclo[7.3.1.0^{2,7}]trideca-2(7),3,5-triene;
5-fluoro-11-aza-tricyclo[7.3.1.0^{2,7}]trideca-2(7),3,5-triene;
6-hydroxy-5-methoxy-11-aza-tricyclo[7.3.1.0^{2,7}]trideca-2(7),3,5-triene and a pharmaceutically acceptable salt and an optical isomer thereof.

31. The method according to claim 30, wherein the nicotine partial agonist is selected from
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
6-methyl-5-thia-5-dioxa-6,13-diazatetracyclo[9.3.1.0^{2,10}.0^{4,8}]pentadeca-2(10),3,8-triene;
4-fluoro-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
6-methyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
11-azatricyclo[7.3.1.0^{2,7}]trideca-2(7),3,5-triene-5-carbonitrile;
1-[l 1-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[l 0.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8} ]hexadeca-2(10),3,6,8-tetraene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol;
and the pharmaceutically acceptable salts and optical isomers thereof.

32. The method according to claim 17, wherein the nicotine receptor partial agonist and the analgesic agent are administered substantially simultaneously.

33. A pharmaceutical composition for treating a disorder or condition selected from the group consisting of diseases and conditions in which pain predominates, including acute pain, chronic pain, neuropathic pain and migraine, and including soft tissue and peripheral damage, such as acute trauma, osteoarthritis, rheumatoid arthritis, musculo-skeletal pain, particularly after trauma, spinal pain, dental pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhea, and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; headache, including migraine, acute or chronic tension headache, cluster headache, temporomandibular pain and maxillary sinus pain; ankylosing spondylitis, gout; post operative pain; and scar pain, in a mammal, including a human, the method comprising administering to said mammal respectively a pain attenuating effective amount of a pharmaceutical composition comprising: (a) a nicotine receptor partial agonist or a pharmaceutically acceptable salt thereof; (b) an analgesic agent or a pharmaceutically acceptable salt thereof and (c) a pharmaceutically acceptable carrier, wherein the active agents “a” and “b” above are present in amounts that render the composition effective in treating acute, chronic and/or neuropathic pain and migraine.

34. A method of treating a disorder or condition selected from the group consisting of diseases and conditions in which pain predominates, including acute pain, chronic pain, neuropathic pain and migraine, and including soft tissue and peripheral damage, such as acute trauma, osteoarthritis, rheumatoid arthritis, musculo-skeletal pain, particularly after trauma, spinal pain, dental pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhea, and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; headache, including migraine, acute or chronic tension headache, cluster headache, temporomandibular pain and maxillary sinus pain; ankylosing spondylitis, gout; post operative pain; and scar pain, in a mammal, including a human, the method comprising administering to said mammal respectively a pain attenuating effective amount of a pharmaceutical composition comprising: (a) a nicotine receptor partial agonist or a pharmaceutically acceptable salt thereof; (b) an analgesic agent or a pharmaceutically acceptable salt thereof and (c) a pharmaceutically acceptable carrier, wherein the active agents “a” and “b” above are present in amounts that render the composition effective in treating acute, chronic and/or neuropathic pain and migraine.
